(19) United States
(12) Patent Application Publication
(10) Pub. No.: US 2004/0181163 A1
(43) Pub. Date: Sep. 16, 2004

(54) Fat analyzer

(75) Inventors: Philip Lim-Kong Wong, Northants (GB); Ka Yiu Sham, Great Falls, VA (US)

Correspondence Address: CROWELL & MORING LLP, INTELLECTUAL PROPERTY GROUP, P.O. BOX 14300, WASHINGTON, DC, 20044-4300, US

(73) Assignee: ACUMEN

(21) Appl. No.: 10/386,535

(22) Filed: Mar. 13, 2003

(51) Int. Cl. A61B 5/05

(52) U.S. Cl. 600/547

(57) ABSTRACT

A four electrode bio-electrical impedance analyzer having a 45 KHz constant current pulse waveform and a synchronized detection scheme to measure the human body impedance and therefore calculate total body resistivity. The resistivity is used in conjunction with the pre-input data concerning characteristics of the person under tests to determine the percentage of body fat in a manner which is repeatable and which suppresses electrical interference.

### BACKGROUND AND SUMMARY OF THE INVENTION

The present invention relates to an improved impedance analyzer or bio-electrical impedance measurement for calculations to related to body fat.

The impedance to electrical flow of an injected current has been found to be a function of the volume of a conductor and the square of the conductor's length. For clinical purposes, bio-electrical impedance measurements make use of this fact to determine a person's impedance to electrical flow based on the volume of the human body and the square of the height of the human. The measurement of volume impedance in a human using a traditional method provides problems with respect to repeatability and electrical noise interference (hum).

It is an object of the present invention to provide a circuit arrangement for establishing volume impedance measurements having improved interference suppression as well as consistent repeatability.

The present invention provides a system of outputting an AC constant current pulse wherein body conductivity is measured in response to the applied pulse to provide a measure of the body fat/body mass ratio.

Other objects, advantages and novel features of the present invention will become apparent from the following detailed description of the invention when considered in conjunction with the accompanying drawings.

### BRIEF DESCRIPTION OF THE DRAWINGS

FIG. 1 is a functional block diagram of the body fat analyzer;

FIG. 2 is a circuit diagram of the constant current pulse source of FIG. 1;

FIG. 3 is a circuit diagram of the measurement and amplification circuit of FIG. 1; and

FIG. 4 is a circuit diagram of the central processor and display unit of FIG. 1.

### DETAILED DESCRIPTION OF THE PREFERRED EMBODIMENT

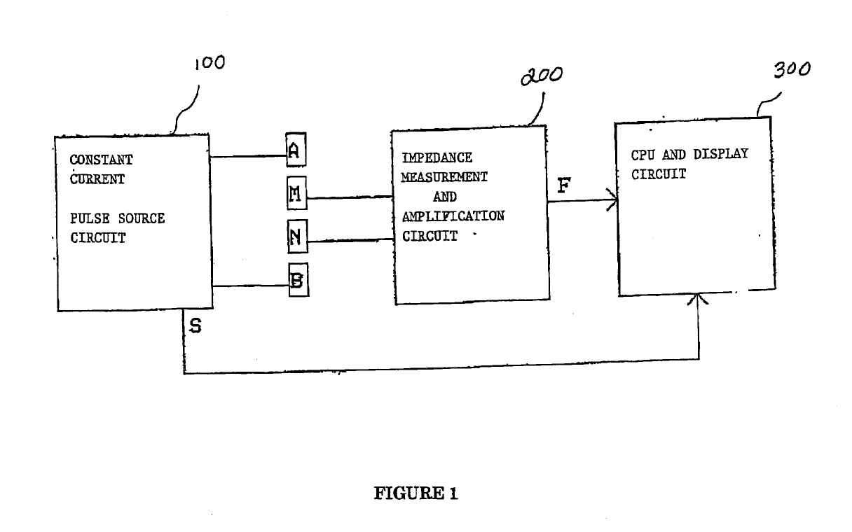

The bio-electrical impedance analyzer (BIA) of FIG. 1 is a four-electrode system which generates 45 KHz constant current pulse waveforms and provides a synchronized detection scheme to measure human body impedance. From this measurement, the total body “resistivity” is computed using pre-input data concerning the body weight, height, age and sex to determine the percentage of body fat to body mass ratio of the person being tested.

The 45 KHz constant current pulse source circuit 100 provides the output to electrode A and B which may be typically located on the respective left and right hand bars of a handgrip device. Current flows between terminals A and B when a person places their right and left hand of the bar to establish contact through contact electrode plates M and N also respectively positioned on the left and right handgrip bars. The motoring takes place by way of the impedance measurement and amplification circuit 200 which provides a measure of the body resistivity. The output measured signal strength F from the impedance and amplification circuit 200 is processed in the CPU and display circuit 300. The output signal strength F is synchronized with the constant current pulse source circuit 100 by way of the output S from the circuit 100 which functions to suppress electrical interference and to improved the ability to repeat measurements on an consistent basis. The microprocessor, using the synchronized measured signal strength determines body fat/body mass ratio.

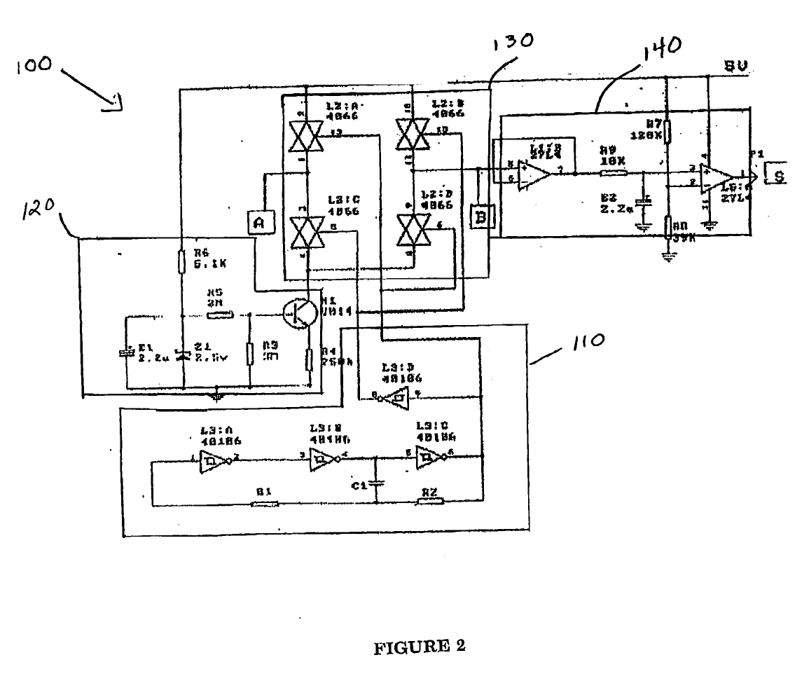

A constant current pulse source circuit 100, in the preferred embodiment is formed by a 45 KHz oscillator 110, the constant current source 120, the polarity switching circuit 130 and the synchronized conditioning and detection circuit 140. The oscillator circuit 110 is formed by oscillator elements L3a, L3b, L3c and L3d as shown in FIG. 2. The constant current source uses the transistor N1 supported by interconnected resistor circuits R3-R6 in conjunction with capacitor E1 and Zener diode Z1. The modulation of the output of constant current source 120 by the oscillator 110 is provided by polarity switching circuit 130 using analogs switch L2a, L2b, L2c and L2d to provide an AC current between the contact electrodes A and B as a stimulant signal. Amplifier circuits L1b and LSN form the conditioned output circuit 140 which output the synchronization signal S.

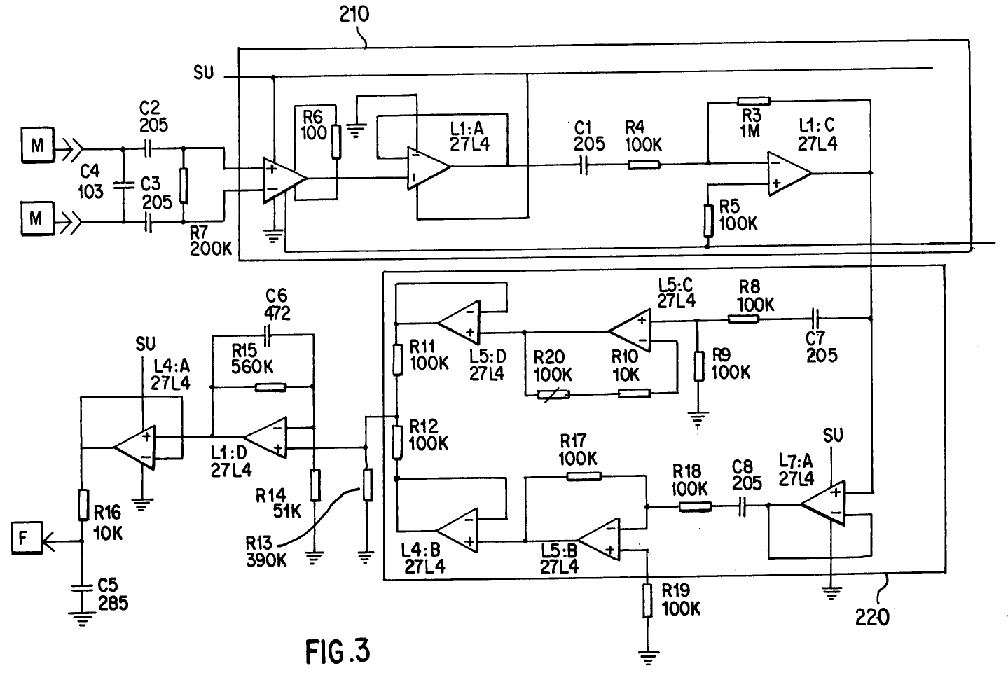

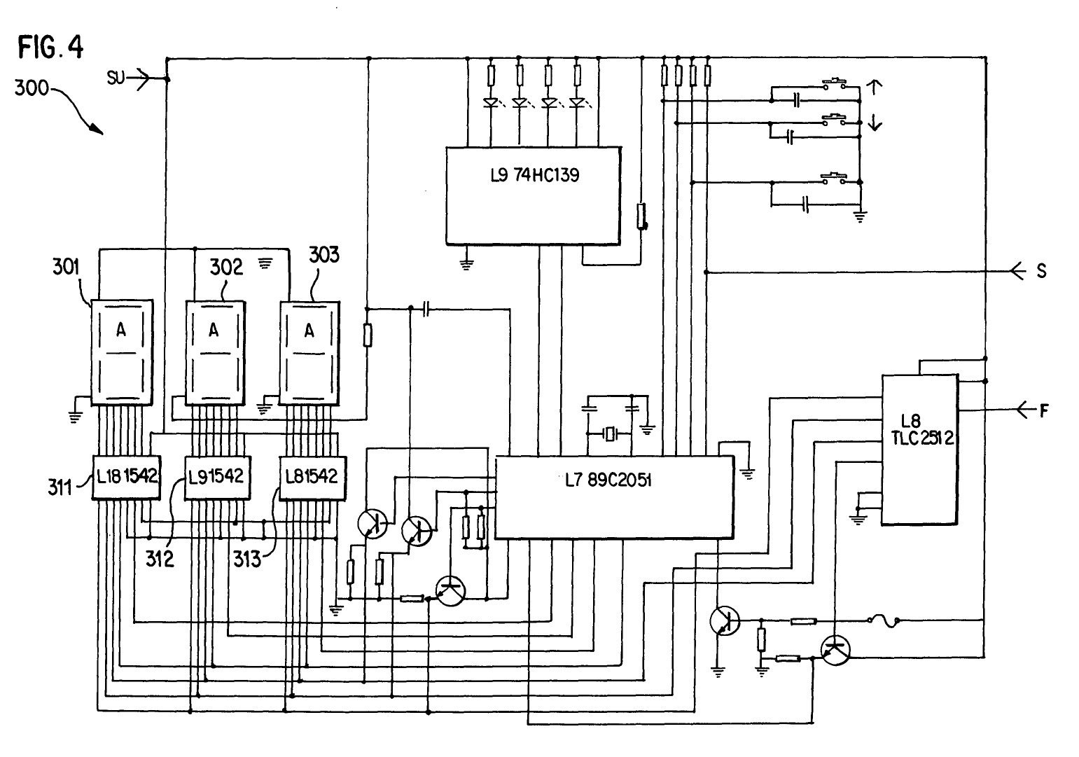

Induced current flow is measured from the contact electrodes M and N by op-amps L, L1a, L1c and L4c in the measuring circuit 210 of FIG. 3. Subsequently the bipolar magnitude signal is modified and combined by rectifier circuit 220 to provide the output signal F as an analog signal to the processor circuit 300 of FIG. 4. Incoming induced current flow signal due to the body resistivity is converted, in processor circuit 300, by the analog/digital converter L8 and is output to the micro-controller L7. The micro-controller L7 is an 8-bit micro-controller which is the main processor for all control and the key pressing functions and display. S1, S2, and S3 are keyinputs. S3 is a Set key used to enter the parameter setup mode. S1 is the UP key to increase a selected parameter and S2 is the Down key to decrease the selected parameter. The display portion includes the LED's 301, 302, 303 and seven-segment drivers 311,312,313. L9 is a 3-8 decoder for converting output to drive the LEDs.

The micro-controller L7 includes pre-input data concerning body weight, height, age and sex of the person holding the handgrip. This pre-input data is combined with the digital magnitude of the induced current flow due to the body resistivity to provide an index for body fat to body mass ratio on the LED display.

Although specific circuitry is used for providing the oscillator 110, the switching circuit 130, the conditioning circuit 140, the constant current source 120 and the measurement circuit 210 and rectifier circuit 220 and well as the analog/digital converter L0, other circuitry known to those skilled in the art for accomplishing the same purpose may be used for each of the specific elements.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

What is claimed is:

1. A bio-electrical impedance analyzer, comprising:
a current pulse source outputting a constant pulse source signal and a synchronizing output signal;
electrode arrangement receiving said pulse source signal;
a measurement circuit for measuring resistivity across said electrode arrangement wherein said measured resistivity is the resistivity of a person in contact with said electrode arrangement; and
a processor circuit for receiving a resistivity output from said measuring circuit and for receiving said synchronized signal output from said pulse source circuit to provide a synchronized output determining the ratio of body fat to body mass of said person.

2. The impedance analyzer according to claim 1, wherein said electrode arrangement includes at least a first pair of contacts on respective right and left handgrips.

3. The impedance analyzer according to claim 1, wherein said current pulse source includes an oscillator circuit, a constant current source and a polarity switching circuit cooperating with said oscillator circuit and said constant current source to provide said constant pulse source signal.

4. The impedance analyzer according to claim 3, wherein said current pulse source further includes a synchronized conditioning and detection circuit for providing said synchronizing signal.

5. The impedance analyzer according to claim 1, wherein said processor circuit includes an analog/digital converter and a micro-controller wherein said micro-controller includes pre-input data concerning body characteristics of said person.

6. A body fat analyzer, comprising:
an electrical source providing a constant current between a first and a second electrode respectively positioned on right and left handgrips;
an electrical impedance measurement device including a third and a fourth electrode respectively positioned on said left and right handgrip for measuring resistivity of a person holding said left and right handgrip;
synchronizing means for receiving the output of said energy source and for outputting a synchronizing signal to a processor circuit for synchronizing the output of said impedance circuit with said energy source to provide an interference free ratio of body fat to body mass of said person.

7. The analyzer according to claim 6 wherein said electrical source comprises a constant current source, an oscillator and a polarity switching circuit to provide said constant current between said first and said second electrodes.

8. The analyzer according to claim 6, wherein said processor circuit include a micro-controller having stored data concerning body, height, age and sex of said person.

9. The analyzer according to claim 6, wherein said synchronizing means includes a conditioning output circuit receiving said energy source output.

10. A method for determining the percentage of body fat in a tested person, comprising the steps of:
applying an output of a constant energy source to a pair of handgrips;
measuring the resistivity of a person holding said handgrips during application of said energy source;
synchronizing said energy source with said resistivity measurement;
storing input data characteristic of said person;
calculating percentage of body fat of said person as a function of said stored data and said measured impedance whereby said measured percentage of body fat is free from electrical interference and provides essentially the same value when said method is repeated for the same tested person.

11. The method for determining percentage of body fat according to claim 10, wherein the step of applying an output of a constant energy source includes the step of switchable modifying an output of a constant current source by means of an output of an oscillator.

12. The method for determining the percentage of body fat according to claim 10, wherein the step of synchronizing said energy source with said resistivity measurement includes the step of conditioning said output of said constant energy source and applying said conditioned signal to a processor wherein said processor also receives said resistivity measurement.

13. The method for determining the percentage of body fat according to claim 10, wherein said step of storing said input data characteristics of said person occurs prior to said step of applying an output of a constant energy source.
